(19) United States
(12) Patent Application Publication
(10) Pub. No.: US 2003/0158251 A1
(43) Pub. Date: Aug. 21, 2003

(54) Methods, pharmaceutical compositions and articles of manufacture for treating disorders associated with abnormal cell proliferation and apoptosis

(76) Inventors: Gad Asher, Ramat Gan (IL); Joseph Lotem, Holon (IL); Batya Cohen, Tel-Aviv (IL); Leo Sachs, Tel-Aviv (IL); Yosef Shaul, M. P. Sde Gat (IL)

Correspondence Address: G.E. EHRLICH (1995) LTD., c/o ANTHONY CASTORINA, SUITE 207, 2001 JEFFERSON DAVIS HIGHWAY, ARLINGTON, VA, 22202, US

(21) Appl. No.: 10/058,042

(22) Filed: Jan. 29, 2002

(51) Int. Cl. A61K 31/366

(52) U.S. Cl. 514/457

(57) ABSTRACT

A method of treating disorders associated with abnormal cell proliferation in a subject in need thereof is provided. The method is effected by administering to the subject a therapeutically effective amount of an agent capable of inhibiting NQO1 activity.

### FIELD AND BACKGROUND OF THE INVENTION

The present invention relates to methods, pharmaceutical compositions and articles of manufacture useful for treating cancer and other cell proliferation associated disorders associated with mutant p53 activity. The present invention is further of a method for regulating a process of apoptosis in a cell associated with wild type p53 activity and a method of identifying potential drugs for treatment of cancer and other disorders associated with abnormal cell proliferation.

The wild-type p53 gene is a tumor suppressor gene. Mutated forms of p53 are found in a variety of tumors (reviewed in refs. 1,2). It is widely accepted that p53 accumulation and activation induces either growth arrest (1,2) or apoptosis (3-6). Typically, p53 does not accumulate in cells because it is a very labile protein, with a half-life as short as a few minutes (7). This lability stems primarily from rapid degradation of p53 in the ubiquitin proteasome pathway. It is widely accepted that p53 plays an important role in cancer development and proliferation, and that the key to effective cancer treatment may lie in control of p53 activity. (Reviewed in; Ryan, K. M., Philips, A. C. and Vousden, K H (2001). Regulation and function of the p53 tumor suppressor protein. Curr Opin Cell Biol 13:332-337).

The accumulation of p53 in response to DNA damage and other types of stress occurs mainly through post-translational modifications. Proteins known to alter p53 stability include HPV16-E6 (8), SV40 large T antigen (9, 10), adenovirus EIB/E4orf6 (11), WT1 (12), and Mdm2 (13, 14). Whereas association of SV40 T antigen or WT1 with p53 increases p53 stability, the binding of E6 or Mdm2 to p53 accelerates its degradation (8-14).

Although these molecular interactions are the subject of numerous scientific publications, none of these molecules have found clinical applications as p53 regulators. Specifically, enhanced p53 degradation and reduction of p53 accumulation and suppression of p53-dependent apoptosis has not been achieved in cells that over-express p53.

Many biological functions of p53 are attributed to its ability to function as a sequence specific transcriptional activator of selected genes (1, 2). One of p53's target genes, PIG3, encodes a protein that shares significant homology with oxidoreductases from several species (15), raising the possibility of p53 regulation by oxidoreductases. This possibility has not been previously investigated and no clinical applications of oxidoreductases as p53 regulators are described.

NADH quinone oxidoreductase 1 (NQO1) is a ubiquitous cytosolic flavoenzyme that catalyzes two-electron reduction of various quinones, utilizing NADH or NADPH as electron donors. This NQO1 mediated reduction mechanism is responsible for the cellular defense against various damaging quinones (16), however some non-toxic quinones such as β-lapachone are reduced by NQO1 to become toxic to cells (17). Expression of the NQO1 gene is induced in response to a variety of agents including oxidants, anti-oxidants and ionizing radiation (reviewed in ref. 18), and NQO1 expression is altered in a number of cancers including breast, colon and lung cancers (19-22). NQO1 is inhibited by dicoumarol [13-3′-methylene bis (4-hydroxycoumarin)], which competes with NADH or NADPH for binding to the oxidized form of NQO1 and thus inhibits NQO1 activity (23). The relationship between NQO1 and p53 has not been previously investigated and no clinical utility for NQO1 or regulators thereof has been suggested.

While numerous patents have been granted on p53 mutations and assays for these mutations as diagnostic methods, relatively few propose use or regulation of p53 as a therapeutic modality.

U.S. Pat. No. 5,770,377 to Picksley et al. teaches a method for interfering with the binding between p53 and MDM2 comprising administering an effective amount of a peptide compound which is able to disrupt or prevent binding between p53 and MDM2, or a functional peptide analogue thereof. Also taught are compounds for use in the method, methods for detecting such compounds and their application in the diagnosis and treatment of tumors. Peptide based drugs have many inherent disadvantages and Picksley neither hints nor suggests dicoumarol or other regulators of NQO1 are able to affect p53 activity or concentration. In fact, the potential utility of NQO1 regulators or inhibitors is not found in these teachings.

U.S. Pat. No. 6,017,524 to Roth et al. teaches methods and compositions for the selective manipulation of gene expression through the preparation of retroviral expression vectors for expressing wild type p53 sequences. Roth does not teach regulation of p53 degradation as a treatment modality. Further, it is not clear if expressing wild type p53 cancels expressing a mutant form of p53 would prove to be an efficacious treatment. Further, Roth neither hints nor suggests dicoumarol or other regulators of NQO1 are able to affect p53 activity or concentration. In fact, the potential utility of NQO1 regulators or inhibitors is not found in these teachings.

U.S. Pat. No. 6,140,058 to Lane et al. teaches mutant forms of p53 protein which are defective in conversion from the latent to the activated state by casein kinase II. Again, Lane does not teach regulation of p53 degradation as a treatment modality. Further, Lane requires activation of the described mutants in order to effect treatment by inducing apoptosis selectively in tumor cells. Further, the potential utility of NQO1 regulators or inhibitors is not found in these teachings.

U.S. Pat. No. 6,143,290 to Zhang et al. teaches compositions and methods involving adenovirus constructs including methods for restoring p53 function and tumor suppression in cells and animals having abnormal p53. Inherent in the teachings of Zhang are all the drawbacks of adenovirus mediated therapy. Further, the potential utility of NQO1 regulators or inhibitors is not found in these teachings.

Thus, while regulation of p53 activity has been a goal of many prior art inventions, the means by which this goal has been pursued are far from optimal. As a result, there is not currently available a reliable means of cancer therapy based upon p53. Further, use of NQO1 as a p53 regulator is not taught by the prior art.

There is thus a widely recognized need for, and it would be highly advantageous to have, methods, pharmaceutical compositions and articles of manufacture which can be used for inhibiting NQO1 activity and thus serve to regulate p53 mediated cellular responses in a variety of disorders.

### SUMMARY OF THE INVENTION

According to one aspect of the present invention there is provided a method of treating cancer and other disorders associated with abnormal cell proliferation in a subject in need thereof. The method comprises administering to the subject a therapeutically effective amount of an agent capable of inhibiting NQO1 activity.

According to another aspect of the present invention there is provided a pharmaceutical composition for treating cancer and other disorders associated with abnormal cell proliferation. The composition includes, as an active ingredient, a therapeutically effective amount of an NQO1 inhibiting agent and a physiologically acceptable carrier and/or excipient.

According to yet another aspect of the present invention there is provided an article of manufacture including packaging material and a pharmaceutical composition identified for treatment of cancer and other disorders associated with abnormal cell proliferation being contained within the packaging material. The pharmaceutical composition includes, as an active ingredient, an agent capable of inhibiting NQO1 activity and a pharmaceutically acceptable carrier.

According to still another aspect of the present invention there is provided a method of regulating apoptosis in a cell, cell culture or tissue, The method comprises contacting the cell, cell culture or tissue with an agent capable of inhibiting NQO1 activity.

According to an additional aspect of the present invention there is provided a method of identifying a drug candidate for treatment of disorders associated with abnormal cell proliferation such as cancer. The method comprises screening a plurality of molecules for a molecule capable of inhibiting NQO1 activity. The molecule capable of inhibiting NQO1 activity is the drug candidate.

According to yet additional aspect of the present invention there is provided a method of identifying an apoptosis inhibitor. The method comprises screening a plurality of molecules for a molecule capable of inhibiting NQO1 activity. The molecule capable of inhibiting NQO1 activity is the apoptosis inhibitor.

According to further features in preferred embodiments of the invention described below, the agent is dicoumarol.

According to still further features in the described preferred embodiments the method further comprises verifying that the abnormal cell proliferation in the subject is associated with a gain of function mutant of p53.

According to still further features in the described preferred embodiments administering is effected via local administration or systemic administration.

According to still further features in the described preferred embodiments administering is effected via a route selected from the group consisting of injection, oral administration, intraocular administration, intranasal administration, transdermal delivery, intravaginal administration and rectal administration.

According to still further features in the described preferred embodiments the therapeutically effective amount is selected such that a concentration of the agent at a site of treatment in the subject is at least 10 μM and no more than 1 mM.

According to still further features in the described preferred embodiments the subject is a human being.

According to still further features in the described preferred embodiments screening is accomplished by measuring at least one parameter selected from the group consisting of NQO1 binding, NQO1 cleavage, NADH binding and binding to a site on a p53 molecule normally bound by NQO1.

According to still further features in the described preferred embodiments screening is effected by at least one method selected from the group consisting of an antibody based assay, an assay for competitive inhibition of NQO1 binding to p53, an assay of inhibition of NQO1 activity, an assay of specific NQO1 binding and an assay of NQO1 molecular weight.

The present invention successfully addresses the shortcomings of the presently known configurations by providing methods, pharmaceutical compositions and articles of manufacture for inhibiting NQO1 activity. This inhibition provides a previously undescribed means for treatment of cancer and other disorders associated with abnormal cell proliferation which relies upon inhibition of NQO1 activity. The present invention is further of a method for regulating a process of apoptosis in a cell by regulating NQO1 activity and a method of identifying a drug candidate by screening for a molecule capable of inhibiting NQO1 activity.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawing

FIGS. 1a-c illustrate dicoumarol induced decrease in p53 levels as detected via immunoblot assays using Pab 1801 monoclonal anti p53 antibody. FIG. 1a-HCT116 cells were incubated without (−) or with 400 micromolar dicoumarol for 90 and 180 min.; FIG. 1b-HCT116 cells were gamma-irradiated at 6 Gy and incubated without (−) or with 200 and 400 micromolar dicoumarol for 4 hrs. This blot was under-exposed relative to that of FIG. 1a in order to highlight the increase in p53 protein level following irradiation; FIG. 1c-COS 1 cells were gamma irradiated at 6 Gy and incubated without (−) or with 200 and 400 micromolar dicoumarol for 4 hrs. Protein levels of each lane probed were calibrated by probing with monoclonal anti beta-tubulin antibody. Dic—Dicoumarol.

FIGS. 2a-b illustrate that proteasomal degradation is responsible for the dicoumarol-induced p53 decrease in cells. FIG. 2a-HCT116 cells were incubated without (−) or with 200 and 400 micromolar dicoumarol and without (−) or with 100 micromolar MG 132 for 4 hrs. FIG. 2b-HCT116 cells were incubated without (−) or with 400 micromolar dicoumarol without (−) or with 40 micromolar lactcystin for 4 hrs. Immunoblots were carried out as in FIG. 1.

FIG. 3 illustrates that dicoumarol-induced p53 degradation is inhibited by over-expression of NQO1. Parental HCT116 cells (−) and a pool of HCT116 stable clones over-expressing HA-tagged NQO1 were incubated without (−) or wit 200 and 400 micromolar dicoumarol for 4 hrs. Immunoblot analysis was carried out using Pab 1801 monoclonal anti p53 antibody and the blots were then stripped and re-probed wit monoclonal anti-HA antibody as a control for NQO1 expression.

FIGS. 4a-c illustrate that dicoumarol inhibits p53 accumulation and p53-dependent apoptosis in gamma irradiated thymocytes. Thymocytes that were not irradiated (−) or gamma irradiated at 4 Gy (+) were cultured for 5 hrs without (−) or with 100 and 200 micromolar dicoumarol. FIG. 4a is a histogram illustrating the percentage of apoptotic cells determined on May-Grünwald Giemsa stained cytospin preparations; FIG. 4b is an ethidium bromide stained gel indicating DNA fragmentation at inter-nucleosomal sites, FIG. 4c is an immunoblot analysis carried out using Pab 240 monoclonal and anti p53 antibody.

FIGS. 5a-b illustrate that dicoumarol mediated decrease of p53 level and p53-dependant apoptosis in M1-t-p53 cells. FIG. 5a—M1-t-p53 myeloid leukemic cells were cultured at 32° C. without or with different concentrations of dicoumarol and the percent of viable cells was determined after 23 hrs. Concentrations of dicoumarol above 125 micromolar were toxic to these cells. FIG. 5b-Immunoblot analysis of p53 level in cells cultured at 32° C. for 16 hrs without (−) or with 75 or 100 micromolar dicoumarol was carried out using Pab 240 monoclonal anti p53 antibody.

FIGS. 6a-b illustrate that degradation of mutant and wild-type p53 is mediated by dicoumarol but not by other anti-apoptotic agents such as interleukin 6 (IL6) or thapsigargin (TG). M1-t-p53 cells were cultured for 6 hrs without (−) or with 10 nanomolar TG, 50 ng/ml IL-6, or 100 micromolar dicoumarol. FIG. 6a illustrates an experiment conducted at 32° C., a condition at which the p53 exhibits wild-type activity. FIG. 6b illustrates an experiment conducted at 37° C., a condition at which the p53 exhibits a mutant activity. Immunoblot analysis was carried out using Pab 240 monoclonal anti p53 antibody. The blots were then stripped and re-probed with anti I-kappa-B and anti beta-tubulin antibody.

FIG. 7 is a model illustrating the role of NQO1 in p53 stabilization. It is assumed that NQO1 determines the level of NAD⁺ and that this regulates the level of p53. The stabilization of p53 results in either apoptosis or growth arrest which regulate life-span. Also shown is the NAD⁺-Sir2p pathway that regulates life-span in yeast.

FIGS. 8a-b are immunoblots illustrating NQO1 activity dependent stabilization of p53 protein. Protein extraction and immunoblot analysis were as described in the Examples section using PAb 1801 monoclonal anti p53 antibody. The blots were then stripped and re-probed with monoclonal anti Ha for the detection of Ha-NQO1 and anti-actin antibody as a control for equal protein loading in each lane. FIG. 8a is an immunoblot of cell extracts from p53 null HCT116 cells transfected with 150 ng PRC/CMV human wild-type p53 with either pSGS empty vector (lane 1), wild-type HA NQO1 (lane 2) or polymorphic HA C609T NQO1 (lane 3). Blots were probed with antibodies indicated on the left. FIG. 8b is an immunoblot of cell extracts from HCT116 (−) and HCT116 stably expressing HA NQO1 (+) cells cultured without any treatments (N.T.; lanes 1+2), γ-irradiated (γ-IR; lanes 3-6) at 6 Gy or treated with 100 μM H₂O₂ (lanes 7+8). Cell extracts were prepared from untreated cells, and from cells cultured for ½ h and 4 h post γ-irradiation and 6 h after addition of H₂O₂. Blots were probed with antibodies indicated on the left.

FIGS. 9a-c are a series of immunoblots which demonstrate that NQO1 partially antagonizes papilloma virus E6 but not Mdm-2 mediated degradation of p53. Immunoblot analysis was carried using Pab 1801 monoclonal anti p53 antibody. The blots were then stripped and re-probed with monoclonal anti Mdm2, anti-Ha for the detection of HA NQO1 and HA LT and anti-Actin antibody as a control for equal protein loading in each lane. In each panel, antibodies employed are indicated to the left. FIG. 9a is an immunoblot of extracts from p53 null HCT116 cells transfected with 150 ng PRC/CMV human wild-type p53 without and with 500 ng PRC/CMV-E6, 2 μg pSG5 wild-type HA NQO1 or 3 μg polymorphic HA C609T NQO1. FIG. 9b is an immunoblot of extracts of p53 null HCT116 cells transfected with 150 ng of PRC/CMV human wild-type p53 without or with 300 ng pCOC-mdm2 X2, 1.5 μg pCGN-HA-LT or 2 μg pSG5 wild-type HA NQO1. FIG. 9c is an immunoblot of extracts of p53 null HCT116 cells transfected with 150 ng of PRC/CMV human wild-type p53 without or with 800 ng PRC/CMV-E6 or 1.5 μg of pCGN-HA-LT.

FIG. 10 is an immunoblot illustrating that LT protects p53 protein from degradation induced by dicoumarol. p53 null HCT116 cells were transfected with 150 ng of PRC/CMV human wild-type p53 without or with 300 ng pCOC-mdm2 X2 or 1.5 μg pCGN-HA-LT. Transfected cells (24 h post transfection) were then cult for 5 h without or with 300 μM dicoumarol. Antibodies used to probe and reprobe the blots are indicated at the left. Stripping and reprobing was as for FIGS. 8 and 9.

FIGS. 11a-b are a series of immunoblots illustrating induction of wild-type and mutant p53 degradation by hsp90 inhibitors. Immunoblot analysis was carried out using Pab 240 monoclonal anti p53 antibody and hamster anti mouse Bcl-2. FIG. 11a is an immunoblot of cell extracts from M1-t-p53 myeloid leukemic cells cultured for 6h at 32° C. or 37° C. without or with different concentrations of radicicol. Antibodies are indicated at left. FIG. 11b is an immunoblot of cell extracts from cells cultured at 32° C. for 6 or 2 h without (−) or with (+) 1 μM radicicol. Cells were also preincubated for 4 h at 32° C. and then cultured with 1 μM radicicol or 1 μM geldanamycin (geldan.) for 2 h (2 h*).

FIG. 12 is a plot of % cell viability as a function of concentration of hsp 90 inhibitor indicating suppression of wild-type p53 mediated apoptosis by hsp90 inhibitors, M1-t-p53 cells were cultured at 32° C. without or with different concentrations of radicicol or geldanamycin. Cell viability was determined 23 h after culture at 32° C.

FIGS. 13a and b are a series of immunoblots illustrating degradation of p53 in 7-M12 myeloid leukemic cells and normal thymocytes treated with dicoumarol, radicicol or Geldan. Immunoblot analysis was carried out using PAb 240 monoclonal anti p53 antibody and rabbit anti IκB FIG. 13a is an immunoblot of extracts from 7-M12 cells which were either not treated (none), γ-irradiated at 0.4 Gy or treated with 2.1 μM doxorubicin (Dox.). Cells were cultured for 2 h without (−) or with 100 μM dicoumarol (Dic.) or 5 μM radicicol. FIG. 13b is an immunoblot of extracts from normal thymocytes which were either not treated (none) or γ-irradiated at 0.4 Gy and cultured without (−) or with 200 μM dicoumarol (Dic.), 5 μM radicicol or 5 μM geldanamycin (Geldan.). Cell extracts were prepared after 2 h and apoptosis was determined after 5 h.

FIG. 14 is a theoretical model of the role of NQO1 in p53 accumulation. The model assumes that the level of p53, depicted as a triangle, is oppositely regulated by Mdm-2 and by NQO1. These pathways function independently with a possible cross talk between them mediated by production of ROS after γ-irradiation (IR). ROS increases the level of NQO1 which in turn reduces ROS by its oxidoreductase activity, in a negative feedback loop. (↓, pathway; ⊥, inhibition of a pathway).

### DESCRIPTION OF THE PREFERRED EMBODIMENTS

The present invention is of methods, pharmaceutical compositions and articles of manufacture for inhibiting NQO1 activity which can be applied to the treatment of cancer and other cell proliferative disorders. Specifically, the present invention can be used to as a novel means for treatment of cancer and other disorders associated with abnormal cell proliferation. The present invention is further of a method for regulating cellular apoptosis and a method of identifying a potential drug candidate for treatment of disorders associated with abnormal cell proliferation (e.g. cancer) by screening for a molecule capable of inhibiting NQO1 activity.

The principles and operation of methods, pharmaceutical compositions and articles of manufacture according to the present invention may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

Because of the generally accepted importance of p53 in cancer progression, a number of prior art patents have dealt with p53 regulation.

Although numerous patents have disclosed various approaches for regulating p53 mediated cellular processes, see for example, U.S. Pat. Nos. 5,770,377; 6,017,524; 6,140,058 and 6,143,290, such approaches generally rely upon p53 expression constructs, mutants or p53 ligands for directly regulating p53 expression and/or activity.

While reducing the present invention to practice, the present inventors have uncovered that inhibition of NQO1 activity specifically and effectively regulates p53 cellular levels.

Thus, while the importance of p53 activity in treatment of cancer has long been postulated, the present invention provides a previously undescribed method for regulating that activity, as well as pharmaceutical compositions and articles of manufacture useful in practice of the claimed method. The invention further provides additional methods for isolating new agents capable of regulating NQO1 activity, and thus regulating p53 levels.

Thus, according to one aspect of the present invention there is provided a method of treating a disorder characterized by p53-associated abnormal cell proliferation in a subject in need thereof, such as a human. Examples of disorders characterized by p53-associated abnormal cell proliferation include, but are not limited to, cell growth associated disorders such as tumors and cancers and disorders associated with early aging of tissues [Cadwell, C. and Zambetti, G P (2001). The effect of wild-type p53 tumor suppressor activity and mutant p53 gain-of-function on cell growth. Gene 277:15-30; Tyner S. D et al (2002), p53 mutant mice that display early ageing-associated phenotypes. Nature, 415:45-53].

The method according to this aspect of the present invention is effected by administering to the subject a therapeutically effective amount of an agent capable of inhibiting NQO1 activity.

According to a preferred embodiment of the present invention, the agent (NQO1 inhibitor) administered is dicoumarol. As is illustrated in the Examples section which follows, dicoumarol is capable of specifically and effectively reducing cellular p53 levels and thus inhibiting aberrant cellular processes mediated by p53. Other NQO1 inhibiting substances include, but are not limited to 5-methoxy-1,2-dimethyl-3[(4-nitrophenoxy)methyl] indole-4,7-dione (ES936; Winski S L et al (2001) Biochemistry 40:15135-42), Ethacrynic acid (Sharma and Jaiswal (1994) Biochem Pharmacol 47:2011-5) and 4,7 dioxobenzothiazoles (Ryu C K et al (2000), Arch Pharm Res 23:554-558).

Administering, according to the method, may be effected locally or systemically. Further description relating to suitable administration routes is provided hereinbelow.

Preferably the therapeutically effective amount administered is selected such that a concentration of the agent at a site of treatment in the subject is at least 10 μM and no more than 1 mM. More preferably the therapeutically effective amount administered is selected such that a concentration of the agent at a site of treatment in the subject is at least 50 μM and no more than 400 μM. Most preferably the therapeutically effective amount administered is selected such that a concentration of the agent at a site of treatment in the subject is at least 25 μM and no more than 400 μM. Preferably the subject upon which the method is practiced is a human being, although the claimed method is expected to find additional utility in animal models.

Preferably, the method described above further includes a step of verifying that the abnormal cell proliferation is associated with a gain of function mutant of p53 prior to administration of the NQO1 inhibitor.

Such verification can be effected by screening a biological specimen obtained from the subject for presence of a p53 gain of function mutant sequence nucleic acid or amino acid sequence). Such screening can be performed, for example, by Southern or Northern probing, by sequencing of p53 PCR products or by single nucleotide restriction fragment polymorphisms (SNIRPS) or other techniques which reveal the presence of, and identity of, small mutations in the p53 locus. Since p53 is well studied and many mutations have been characterized, one ordinarily skilled in the art will be capable of ascertaining from results of, for example, SNIRPS analysis, whether a gain of function mutant is present.

The NQO1 inhibitor can be administered per se or in a pharmaceutical composition where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intravenous, intraperitoneal, intranasal, or intraocular injections.

Alternately, one may administer the pharmaceutical composition in a local rather than systemic manner, for example, via injection of the is pharmaceutical composition directly into a tissue region of a patient.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical composition for use in accordance with the present invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition (i.e., the agent capable of inhibiting NQO1 activity) may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active ingredient with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatine as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilize. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredient for use according to the present invention is conveniently delivered in the form of an aerosol spray presentation from a pressurized pack or a nebulizer with the use of a suitable propellant, e.g., dichlorodiflaoromethane, trichlorofluoromethane, dichloro-tetrfluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of the present invention may also be formulated in rectal compositions such as suppositories or retention enemas, using, e.g., conventional suppository bases such as cocoa butter or other glycerides.

Pharmaceutical compositions suitable for use in context of the present invention include compositions wherein the active ingredients are contained in an amount effective to achieve the intended purpose. More specifically, a therapeutically effective amount means an amount of active ingredients (e.g. dicoumarol) effective to prevent, alleviate or ameliorate symptoms of a disorder (e.g., tumor progression) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patients condition. (See e.g., Fingl, et al., 1975, in “The Pharmacological Basis of Therapeutics”, Ch. 1 p. 1).

Dosage amount and interval may be adjusted individually to provide levels of the active ingredient which are sufficient to suppress cell proliferation at the site of treatment (minimal effective concentration, MEC). The MEC will vary for each preparation, but can be estimated from the data provided herein. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the abnormal state is achieved.

The amount of a composition to be administered will, of course, be dependent on the subject being treated, the severity of the affliction, the manner of administration, the judgment of the prescribing physician, etc.

In addition, the pharmaceutical compositions may further include conventional cancer therapy agents, including but not limited to, steroid hormones, cytotoxic chemicals and agents which damage DNA.

Compositions of the present invention may, if desired be presented in a pack or dispenser device, such as an FDA approved kit, which may contain one or more wait dosage forms containing the active ingredient. The pack may, for example, comprise metal or plastic foil, such as a blister pack. The is pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as if further detailed above.

The agent capable of inhibiting NQO1 activity and compositions including such an agent can also be used to regulate apoptosis in a cell, cell culture or tissue since it is well known that wild type p53 activity is associated with cellular apoptosis.

Regulation of apoptosis in a cell, cell culture or tissue is expected to find utility in a wide range of applications including, but not limited to, study of physiologic processes involved in apoptosis and/or cell proliferation pathways. Specifically, laboratory studies of pathological apoptosis and cachexia are expected to benefit from implementation of the present invention.

According to an additional aspect of the present invention there is provided a method of identifying a drug candidate for treatment of disorders associated with abnormal cell proliferation including, but not limited to, cancer.

The method according to this aspect of the present invention is effected by screening a plurality of molecules for a molecule capable of inhibiting NQO1 activity.

Following screening, suitable candidates can be tested in cell cultures and test subjects in order to assess the treatment potential thereof with respect to cancer and other disorders associated with abnormal cell proliferation.

Screening for suitable candidates can be accomplished, for example, by measuring at least one parameter such as NQO1 binding, NQO1 cleavage, NADH binding and binding to a site on a p53 molecule normally bound by NQO1. Therefore, screening may be accomplished using a variety of assays, including, but not limited to, an antibody based assay, an assay for competitive inhibition of NQO1 binding to p53, an assay of inhibition of NQO1 activity, an assay of specific NQO1 binding and an assay of NQO1 molecular weight.

As an illustrative example, known proteolytic enzymes might be screened for their ability to specifically cleave NQO1. This could be achieved, for example, by preparing a crude cell extract containing NQO1 and other cellular proteins. Aliquots of the extract could then be incubated with various enzymes for different periods of time. Analysis of NQO1 cleavage would be by SDS-PAGE followed by immuno-blotting with an NQO1 specific antibody. Methods of preparing such an antibody are known to those skilled in the art and preparation of an anti-NQO1 antibody is described hereinbelow. Specificity of cleavage can be ascertained by probing with additional antibodies such as anti beta-tubulin as described hereinbelow.

As an additional illusive example, molecules might be screened for their ability to specifically bind NQO1. One method of performing such a screen is to prepare radiolabelled NQO1, for example by introducing S³⁵ methionine into a cell free translation reaction using NQO1 mRNA. Potential binding compounds covalently bound to a suitable substrate (e.g. agarose or sepharose) are then incubated with the NQO1-S³⁵ and repeatedly washed. Assays of radioactive disintegration of S³⁵ bound to the substrate serve to indicate which of the screened compounds have the greatest NQO1 binding activity.

The ability to accurately and rapidly screen for NQO1 inhibition enables large scale applicability of the method according to this aspect of the present invention. Thus, the present methodology is amenable to high throughput screening and as such, it is expected that several other NQO1 inhibitors will be uncovered by the present methodology.

It will be appreciated that the above described screening method can also be employed for identifying apoptosis inhibitors

Mutations in p53 are found in more than 50% of the cases in human cancer (1,2). Many of these p53 mutants are gain of function mutants, which can suppress apoptosis (37-39). The ability of an NQO1 inhibitory agent, such as, for example, dicoumarol, to induce degradation of p53 in its mutant form suggest that treatment of these cancers is, by use of the present invention, now feasible.

Dicoumarol is already in clinical use as an anti-coagulant so that it is generally considered a “safe” drug. Thus, dicoumarol is expected to find widespread utility, either alone, or in combination with cytotoxic agents, in therapy against cancer cells that express high levels of mutant p53.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the clam section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,014; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols; A Guide To Methods And Applications”. Academic Press, San Diego, Calif. (1990); Marshak et al., “Strategies for Protein Purification and Characteriztion—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### General Materials and Methods

Cells and Cell Culture:

The cell lines used were: HCT116 human colon carcinoma cells, HCT116 HA-NQO1 over-expressing cells (45), p53 null HCT116 cells (Bunz, F., Dutiaux, A., Lengauer, C., Waldman, T., Zhou, S., Brown, J. P., Sedivy, J. M., Kinzler, K. W. & Vogelstein, B. (1998) Science 282, 1497-1501.), COS 1 monkey kidney cells, normal thymocytes obtained from 2.5 month old Balb/C mice and M1-t-p53 mouse myeloid leukemic cells that express a temperature-sensitive mutant p53 [Val-135] protein (Yonish-Rouach, E., Resnitzky, D., Lotem, J., Sachs, L., Kimchi, A. & Oren, M. (1991) Nature 352, 345-347).

The p53 in M1-t-p53 cells behaves like a tumor-suppressing wild-type p53 at 32° C. and like a mutant p53 at 37° C. (24). HCT116 and COS 1 cells were grown in Dulbecco's modified Eagle's medium (DMEM) supplemented with 10% fetal bovine serum (FBS), 100 units/nl penicillin, and 100 g/ml streptomycin and cultured at 37° C. in a humidified incubator with 5.6% C02. Normal thymocytes and M1-t-p53 cells were grown in DMEM supplemented with 10% heat inactivated (56° C., 30 min) horse serum and cultured at 37° C. in an incubator with 10% C₂.

Chemical: Dicoumarol was obtained from Sigma Chemical Co., St. Lois Mo. and dissolved in 0.13N NaOH, MG132 and lactcystin (Sigma Chemical) were dissolved in DMSO. Radicicol and geldanamycin (Calbiochem) were dissolved in DMSO.

Establishment of HA-NQO1 over-expressing cell lines:

The coding region of the human NQO1 (genbank accession no. J03934) including a 5′ Influenza Hemeagglutinin (HA) tag was inserted into the pEFIRES expression vector containing a puromycin resistance gene (25). HCT116 cells (2.5×10⁶ cells, in 10-cm plates) were transfected with 10 micrograms of purified pEFIRES-HA-NQO1 plasmid using the Superfect transfection reagent (Qiagen, Valencia, Calif., USA). Puromycin resistant colonies expressing HA-NQO1 were identified by immunoblot analysis using anti HA antibody.

Plasmids:

The plasmids used were: PRC/CMV human p53 (47), pEFIRES HA-NQO1 (45.), pCOC-mouse mdm2 X2 (47), PRC/CMV-E6 and pCGN-HA-LT (obtained from U. Nudel). The C609T polymorphism in NQO1was generated by site-directed mutagenesis as described in examples hereinbelow The wild-type HA-NQO1 and the C609T polymorphic HA-NQO1 were cloned into the pSG5 vector and their sequence was verified by DNA sequencing.

Transfection: Cells were seeded at 60% confluence in 6-well plates 16 h before transfection with the desired plasmids. Transfections were carried out by the calcium phosphate method followed by a 10% glycerol shock for 30 seconds, 7 h post transfection. The exact amount of plasmid used in each experiment is indicated in the corresponding figure legend. Whenever needed an empty vector was used to maintain a constant amount of 5 μg total DNA in each transfection mix. Cell extracts were generally prepared 24 h after transfection.

Immunoblot Analysis:

Cell extracts were prepared by lysis of PBS-washed cells in RIPA lysis buffer [150 mM NaCl, 1% NP-40 (vol/vol), 0.5% AB-deoxycholate (DOC vol/vol), 0.1% SDS (vol/vol), 50 mM Tris-Hcl pH 8, 1 mM dithiotireitol (DTT) and 1 micogram/ml each of leupeptin, aprotinin, pepstatin (Sigma cocktail)]. The insoluble pellet was discarded and protein concentration was determined using Bradford reagent (BioRad). Equal amounts of protein were mixed with Laemmli sample buffer (4% SDS, 20% glycerol, 10% 2-mercaptoeftanol and 0.125M Tris-Hcl), heated at 95° C. for 5 min and loaded on an 8% polyacrylamide-SDS gel. Following electophoresis, proteins were transferred to cellulose nitrate 0.45 micrometer membranes (Schleicher & Schuell, Dassel, Germany). Loading equivalence and transfer efficiency were monitored by Ponceau S staining and the membranes were then incubated with appropriate antibodies to proteins of interest followed by horse-radish peroxidase conjugated anti-IgG antibodies. Signals were developed using Super Signal (Pierce Poxkford, USA) at 20° C. for 5 min and the membranes were then exposed to X-ray film (Fuji Tokyo, Japan) for an appropriate time and developed. Membranes were stripped using 50 mM citric acid before using a different primary antibody. The antibodies used were; Monoclonal anti human p53 (Pab 1801) (26), monoclonal anti mouse and human p53 (Pab240), monoclonal anti I-kappa-B (Santa Cruz Biotechnology, Santa Cruz, Calif., USA), monoclonal anti beta-tubulin and anti HA (Sigma Chemical, St. Louis, Mo.).

Apoptosis and Cell Viability Assays:

Apoptosis in normal thymocytes was induced by gamma irradiation 4 Gy (Co⁶⁰ source 0.63 Gy/min) and in M1-t-p53 cells by culture at 32° C. The percentage of apoptotic thymocytes was determined on May-Grünwald-Giemsa-stained cytospin preparations by counting 400 cells 5 hrs post gamma-irradiation. Apoptotic cells were scored by their smaller size, condensed chromatin, and fragmented nuclei compared with non-apoptotic cells. Analysis of DNA fragmentation during apoptosis in thymocytes was performed by DNA agarose gel electophoresis as described (27). Apoptotic M1-t-p53 cells undergo secondary changes including uptake of trypan-blue (28). The percent of viable cells (non apoptotic and not stained with trypan-blue) was determined by counting 400 cells in a hemocytometer after 23 hrs at 32° C.

### Example 1

### Assay of Regulation of p53 Degradation by NQO1

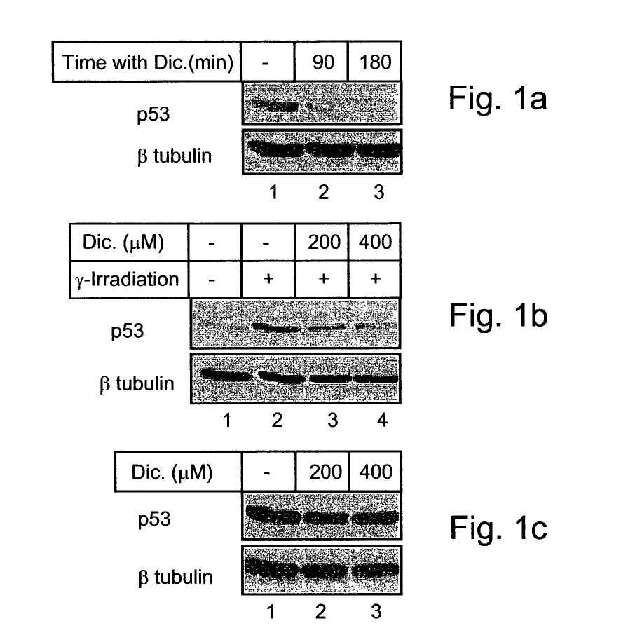

In order to determine whether p53 level is regulated by NQO1, human colon carcinoma cells expressing wild-type p53 (HCT1 16; reference 29), were treated with the NQO1 inhibitor dicoumarol. Treatment for 90 min resulted in a significant reduction in p53 level as evidenced by immunoblot analysis of cell extracts with an anti-p53 antibody. Treatment for 180 min resulted in almost complete elimination of p53 (FIG. 1a). Blots were stripped and re-probed with monoclonal anti beta-tubulin antibody as a control for equal protein loading in each lane.

Since it is known that irradiation cause accumulation of p53 within cells, an additional event was conducted on irradiated cells. HCT-116 cells were γ-irradiated at 6 Gy and incubated for 4 hrs without or with dicoumarol. As expected, p53 accumulated upon γ-irradiation (compare FIG. 1b lanes 1 and 2). Accumulation of p53 was reduced by the presence of 200 micromolar dicoumarol and further reduced at 400 micromolar dicoumarol (FIG. 1b). Under the same conditions the level of beta-tubulin was unaffected (FIG. 1a and b lower panels), indicating that the observed effect is p53 specific.

In order to verify that the observed effect of dicoumarol on p53 level was specific, COS 1 cells expressing SV40 large T antigen which stabilizes p53 (30) were employed in an additional experiment. As expected, the p53 level in COS 1 cells was not reduced by treatment with dicoumarol even at 400 micromolar FIG. 1c).

Together, these results indicate that dicoumarol causes a strong decrease in both basal and induced p53 levels. Since the NQO1 inhibitor does not overcome stabilization of p53 by SV40 large T antigen, it seems that dicoumarol blocks p53 protein stabilization by NQO1.

### Example 2

### Induction of Proteasomal Degradation by an NQO1 Inhibitor

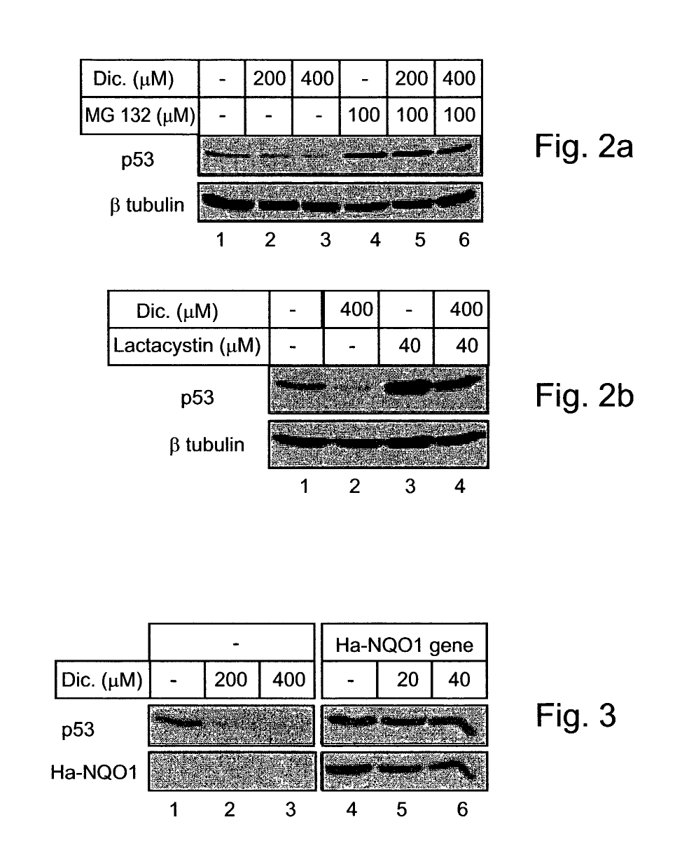

It is well documented that p53 accumulation is determined by the rate of its proteasomal degradation (reviewed in refs. 1,2). In order to determine whether the observed p53 destabilization by dicoumarol occurs through protein degradation, cells were treated with dicoumarol together with the proteasome inhibitors MG 132 or lactacystin. Immunoblot analyses with anti p53 antibodies reveal that dicoumarol-induced p53 elimination was completely blocked by addition of either MG132 (FIG. 2a) or lactacystin (FIG. 2b). These results indicate that dicoumarol induced p53 degradation is via a proteasomal mechanism.

### Example 3

### Over Expression of NQO1 Blocks Dicoumarol

### Mediated p53 Degradation

In order to positively establish that p53 degradation observed in response to dicoumarol was due exclusively to inhibition of NQO1 activity, over-expression of NQO1 in cells was undertaken. A pool of stable clones of cells over-expressing HA-tagged NQO1 was established and the level of NQO1 protein was verified by immunoblotting. These cells became resistant to p53 degradation by dicoumarol FIG. 3). Similar results were obtained with 3 individual stable clones expressing HA tagged NQO1. The level of HA-NQO1 was not reduced in the presence of dicoumarol (FIG. 3). These results indicate that dicoumarol-induced p53 degradation is the direct outcome of inhibition of NQO1 activity.

### Example 4

### Suppression of p53 Mediated Apoptosis by Dicoumarol-Induced p53 Degradation

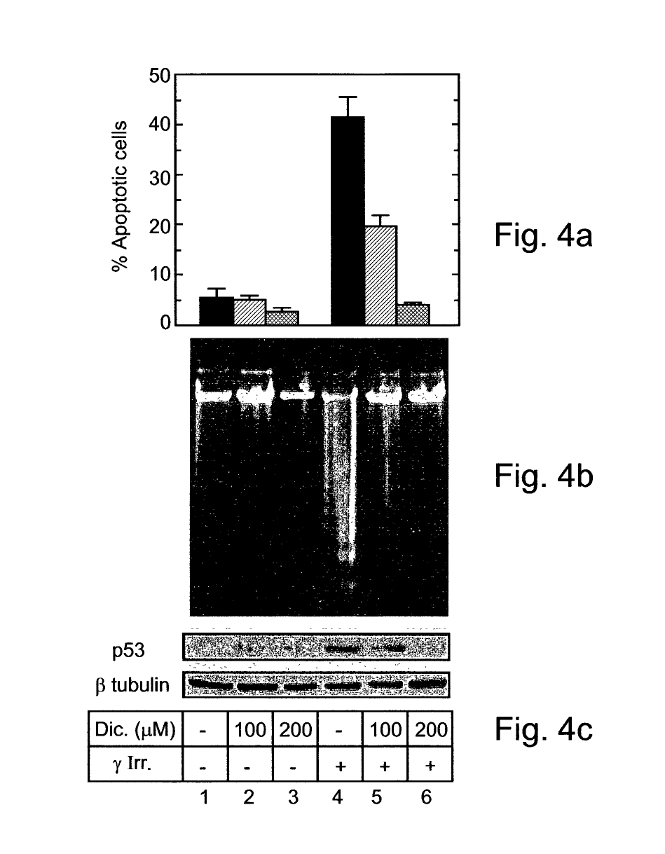

Induction of wild-type p53 accumulation in various cell types either by over-expression of p53, or following gamma irradiation can lead to apoptotic cell death (3-6). Since inhibition of NQO1 by dicoumarol blocks p53 protein accumulation, it seemed likely that dicoumarol would be useful as an apoptosis regulator. In order to verify this possibility, the effect of dicoumarol on p53 dependent apoptosis in gamma irradiated normal mouse thymocytes was assayed. Dicoumarol inhibited induction of apoptosis in 4 Gy gamma irradiated thymocytes in a dose dependent manner, as determined by morphological analysis of apoptosis (FIG. 4a) and DNA fragmentation at inter-nucleosomal sites (FIG. 4b). Complete inhibition of apoptosis was obtained with 200 micromolar dicoumarol. The inhibition of p53-dependent apoptosis in gamma irradiated thymocytes by dicoumarol was associated with a decrease in the level of p53 (FIG. 4c). These results indicate that dicoumarol inhibits p53-mediated apoptosis in gamma irradiated normal thymocytes through enhanced p53 degradation.

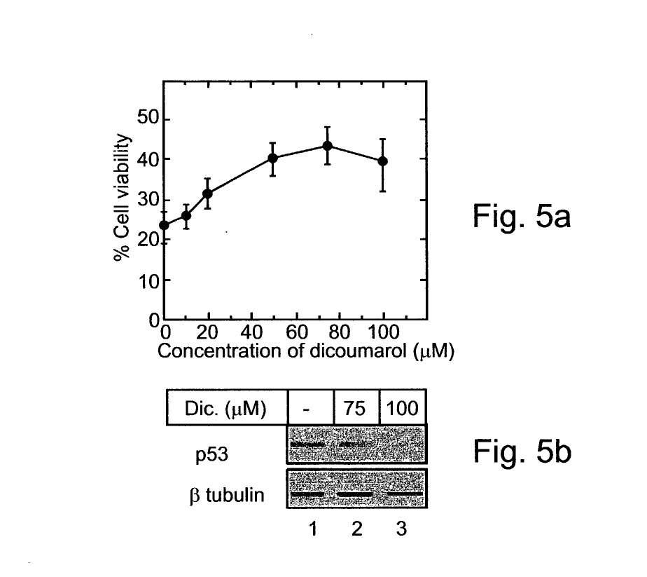

The ability of dicoumarol to affect induction of apoptosis in M1-t-p53 myeloid leukemic cells that over-express a temperature-sensitive p53 transgene was also assayed. These cells are viable and proliferate at 37° C. when the p53 behaves like a mutant form, but undergo apoptosis at 32° C. when the p53 behaves like wild-type (3). Dicoumarol inhibited p53-induced apoptosis in these cells. The addition of 75 μιχρoμoλαρ or 100 micromolar dicoumarol at 32° C. for 23 hrs resulted in an increase in cell survival compared to cells cultured under the same conditions without dicoumarol (FIG. 5a). Doses of 125 micromolar dicoumarol or more were toxic to these cells. As in HCT116 cells and normal thymocytes, p53 levels in M1-t-p53 cells cultured at 32° C. were reduced by addition of dicoumarol (FIG. 5b). Thus, inhibition of NQO1 activity enhanced degradation of over-expressed p53 and resulted in reduced p53-dependant apoptosis in these cells.

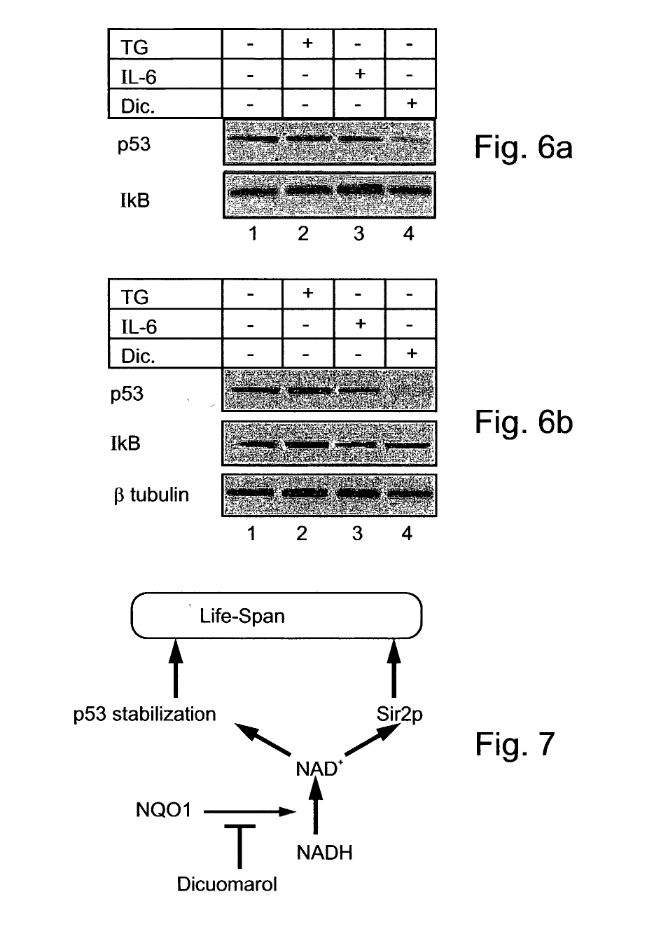

Because interleukin 6 (IL-6) and the calcium mobilizing compound thapsigargin (TG) are known to be efficient anti-apoptotic agents in M1-t-p53 cells (3, 31), the effect of these compounds on p53 stability was also investigated. In contrast to dicoumarol, IL6 and TG did not cause a reduction in p53 level in these cells at 32° C. (FIG. 6a). Dicoumarol did not affect the level of I-kappa-B (FIG. 6a lower panel). The ability of dicoumarol to induce degradation of p53 but not of I-kappa-B or beta-tubulin was also observed in M1-t-p53 cells at 37° C., where the cells express a high level of mutant p53 (FIG. 6b). These results clearly indicate that the effect of dicoumarol on apoptosis is through its effect on NQO1.

### Example 5

### A Model Regulation of Life Span by Dicoumarol and NQO1

FIG. 7 is a schematic representation of the mechanism by which dicoumarol and NQO1 exert opposite regulatory effects on life span (cellular and whole tissue). As pictured, the oxidoreductase activity of NQO1 is mediated by the conversion of NADH to NAD⁺. Therefore, inhibition of NQO1 by dicoumarol causes a substantial NAD⁺ loss. Human breast, skin, and lung cells with reduced NAD level due to the use of nicotinamide deficient medium, exhibit decreased basal levels of p53 protein (32, 33). Recently, NAD has been shown to play an important role in regulation of gene expression and life span in yeast (34-36). A major gene in this process is Sir2p that possesses ADP-ribosyltransferase activity and promotes silencing of gene transcription at selected loci (34, 35). In yeast increased longevity can be induced by calorie restriction and this requires Sir2p and one of the two major pathways of NAD synthesis (36). Sir2p and p53 are structurally and functionally distinct, and our results suggest that both share the capacity to regulate cell fate via NAD (FIG. 7).

### Example 6

### Enzymatic Activity of NQO1 is Responsible for Stabilization of p53

In order to demonstrate that the enzymatic activity of NQO1 is required for p53 stability, a mutant NQO1 expression vector with a C-to-T base pair substitution at position 609 of NQO1 cDNA (C609T) was prepared. This mutation is a genetic polymorphism in humans that results in a proline-to-serine substitution at residue 187 associated with loss of enzyme activity (48).

This was accomplished by site-directed mutagenesis of HA-NQO1 performed by PCR using PWO Taq DNA polymerase (Boehringer Mannheim) and the following primers:

5′-CAAGTCTTAGAATCTCAACTGACAT-3′(sense; SEQ ID NO:1);

3′-ATGTCAGTTGAGATTCTAAGACTTG-5′(antisens; SEQ ID NO:2);

5′- ATAAGATCTATGOCATATCCATATGATGTGC-3′(vector primer sense SEQ ID NO:3); and

3′-ATAAGATCTGGATCCTCATTTTCTAGCTTTG-5′(vector primer antisense, SEQ ID NO:4).

The wild-type HA-NQO1and the C609T polymorphic HA-NQO1 were cloned into the pSG5 vector and their sequence was verified by DNA sequencing as described hereinabove.

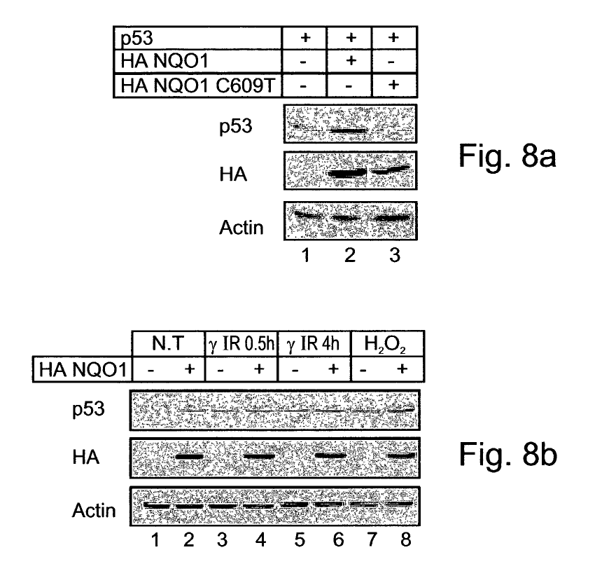

Co-transfection of p53 null HCT116 cells (46) with wild-type p53 and wild-type NQO1 expression vectors stabilized the level of transfected p53 protein (FIG. 8a lanes 1,2). Unlike the wild-type NQO1, C609T polymorphic NQO1 did not stabilize the co-transfected p53 (FIG. 8a lanes 1-3). The level of C609T NQO1 expressed in the transfected cells was lower than the wild type NQO1 (FIG. 8b lanes 2,3). This was despite repeated attempts to increase the level of C609T NQO1 expression, It is postulated that this failure is due to the fact that the mutant protein is highly unstable (33). These results clearly indicate that stabilization of p53 by NQO1 depends on the oxidoreductase activity of the enzyme. This dependence on enzymatic activity is unique among p53 regulators.

### Example 7

### NQO1 Stabilizes p53 Protein Under Oxidative Stress

The effect of transfected NQO1 on endogenous p53 levels under normal conditions and oxidative stress was compared. Under normal growth conditions NQO1 stably transfected HCT116 cells exhibited an elevated level of p53 relative to nontransfected cells (FIG. 8b lanes 1,2).

Oxidative stress was induced by γ-irradiation (assay 30 minutes post treatment) or by administration of H₂O₂ (assay 4 hrs post treatment). Under oxidative stress conditions, NQO1 transfected cells have the same level of p53 as the nontransfected cells. (FIG. 8b lanes 2,3 and 7). The p53 stabilizing effect of NQO1 was most prominent in cells that were exposed to H₂O₂ (FIG. 8b lanes 7,8). These results indicate that NQO1 interferes with an inherent pathway that leads to p53 degradation, especially under oxidative stress, possibly through Mdm2.

### Example 8

### NQO1 Interference with the Effect of Overexpressed Mdm-2 or E6

Because the cellular protein Mdm-2 and the human papilloma virus protein E6 are known to promote ubiquitination and proteasomal degradation of wild-type p53 (reviewed in refs. 2 and 40). The ability of NQO1 to interfere with the effect of overexpressed E6 or Mdm-2 on p53 degradation was assayed in order to better understand the mechanism of NQO1 mediated stabilization of p53.

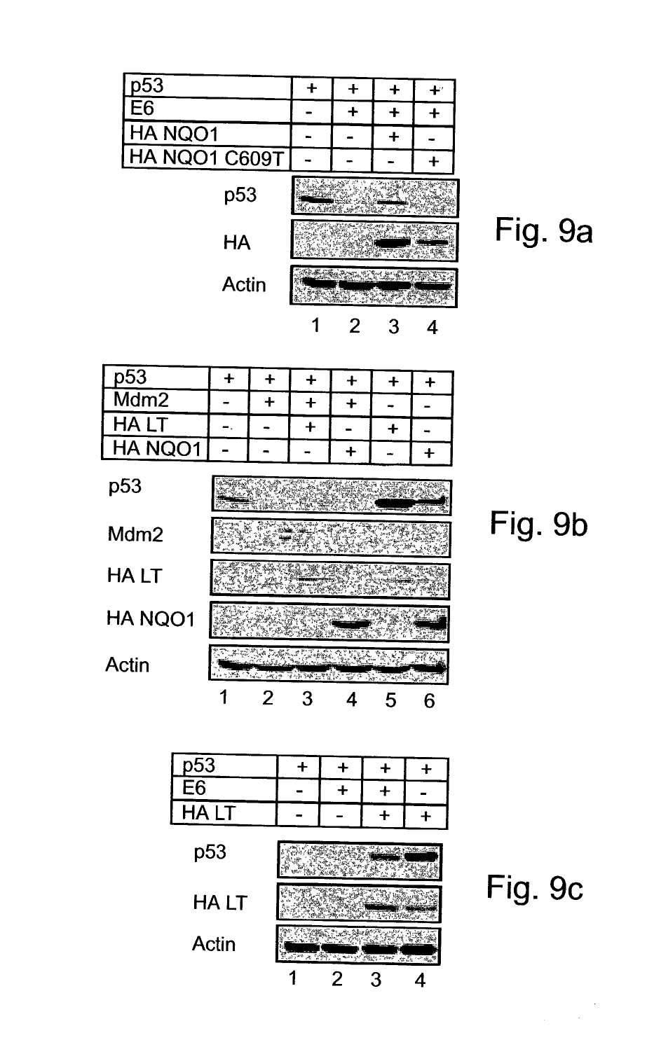

P53 null HCT116 cells were transiently transfected with wild-type p53 alone or co-transfected with different combinations of p53, E6, Mdm-2, wild-type NQO1 and the C609T polymorphic NQO1 (described hereinabove). E6 mediated p53 degradation was partially inhibited by co-transfected wild-type NQO1 but not by the C609T polymorphic NQO1 (FIG. 9a).

In contrast, although NQO1 stabilized p53 (FIG. 9b lanes 1,6) it did not inhibit the effect of overexpressed Mdm-2 on p53 degradation (FIG. 9b lanes 2,4). Thus, NQO1 mediated p53 stabilization is via a pathway that is distinct from that of Mdm2.

The NQO1 inhibitor dicoumarol induces p53 degradation in various cell types, but not in COS-1 cells that express LT (45.). This is because p53 stabilization by LT prevents degradation when NQO1 activity is reduced. In order to demonstrate that this effect is not cell specific, p53 null HCT116 cells were transiently transfected with different combinations of wild-type p53, Mdm-2, E6, NQO1 and LT. LT transfection caused greater elevation of p53 levels than NQO1 transfection (FIG. 9b lanes 1,5,6). Unlike NQO1, LT partially inhibited the ability of Mdm-2 to promote p53 degradation (FIG. 9b lanes 3,4). Further, LT was more potent than NQO1 in antagonizing the p53 degradation effect of E6 FIGS. 9a and c lanes 2,3).

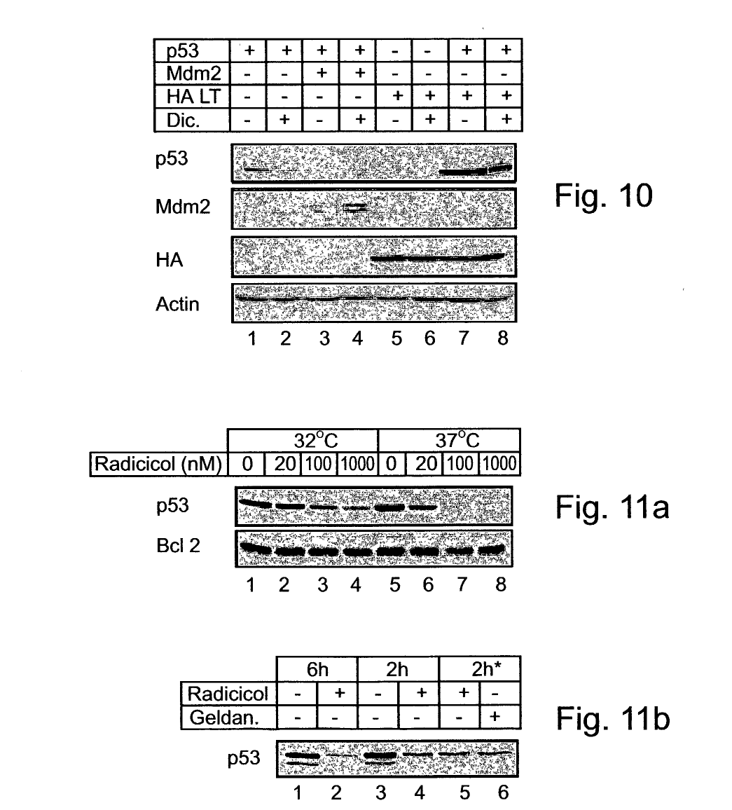

HCT 116 cells co-transfected with p53 and LT were resistant to dicoumarol induced p53 degradation FIG. 10 lanes 7,8), whereas cells transfected only with p53 were sensitive to dicoumarol induced p53 degradation (FIG. 10 lanes 1,2). The levels of both Mdm-2 (FIG. 10 lanes 3,4) and LT (FIG. 10 lanes 5-8) were not affected by dicoumarol. This indicates that dicoumarol affected neither transfection efficiency nor the stability of Mdm-2 and LT.

These results clearly demonstrate that both LT and NQO1 stabilize p53 and block dicoumarol mediated p53 degradation (7). However, unlike LT, NQO1 does not affect p53 degradation mediated by overexpressed Mdm-2. This confirms that the NQO1 pathway is separate and distinct from the Mdm-2 pathway.

### Example 9

### NQO1 and hsp90 Stabilize p53 by Different Mechanisms

It has been previously determined using different types of tumor cells carrying wild-type or mutant p53 that hsp90 interacts with mutant p53 but not with wild-type p53 and that hsp90 inhibitors could disrupt this interaction and cause degradation of mutant p53 (41, 42, 43, 44 and 49). It has further been demonstrated using the ras transformed rat embryo fibroblast cell line A1 which expresses the temperature sensitive Val¹³⁵ mutant p53, that hsp90 inhibitors can also cause degradation of wild-type conformation p53 at 32° C. (43).

The same temperature sensitive Val¹³⁵ mutant p53 is degraded in its mutant and wild-type forms in M1-t-p53 myeloid leukemic cells in the presence of the NQO1 inhibitor dicoumarol (45). This observation raised the possibility that NQO1 and hsp90 inhibitors may induce p53 degradation in certain cell types through a similar mechanism.

In order to ascertain whether a shared mechanism exists, M1-t-p53 leukemic cells were employed to determine whether hsp90 inhibitors can also cause degradation of p53 in its mutant form at 37° C. and its wild-type form at 32° C. Results indicate that a 6 h incubation of M1-t-p53 cells with the hsp90 inhibitor radicicol caused a strong decrease in the level of p53 both in its mutant form (at 37° C.) and in its wild-type form (at 32° C.) (FIG. 11a). The decrease at 37° C. appeared to be stronger than at 32° C. Under the same conditions there was no change in the level of Bcl-2 (FIG. 11a), indicating that the radicicol induced decrease in p53 level was not due to a general effect on the stability of all cellular proteins. To ensure that the radicicol induced decrease in p53 in cells shifted to 32° C. was not due to a rapid degradation of mutant p53 before it could assume a wild-type conformation, cells were preincubated at 32° C. for 4 hr and prior to addition of radicicol. Incubations of as little as 2 h with radicicol at 32° C. were sufficient to cause a decrease in p53 level, irrespective of whether radicicol was added at the time of shift from 37° C. to 32° C. or 4 h after the temperature shift (FIG. 11b lanes 3-5).

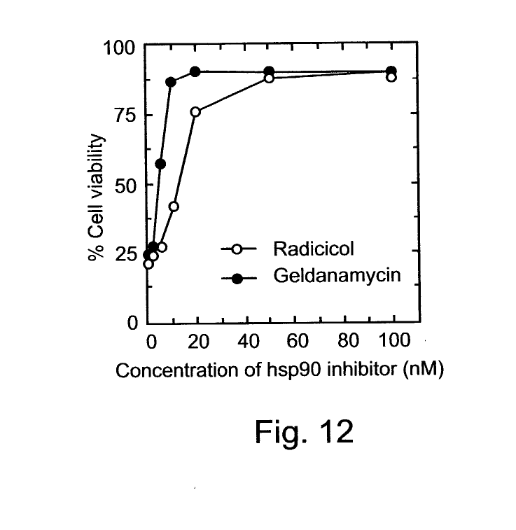

Another hsp90 inhibitor, geldanamycin, produced the same decrease in p53 level at 32° C. (FIG. 11b lane 6). Both radicicol and geldanamycin strongly suppressed the ability of the wild-type p53 to induce apoptosis in the M1-t-p53 myeloid leukemic cells when cultured at 32° C. FIG. 12). Cell viability at 32° C. reached 90±4% in the presence of 100 nM radicicol or geldanamycin (FIG. 12) and was thus more effective than dicoumarol, that at the optimum concentration of 75 μM increased cell viability only to 52±4% (17).

Effects of dicoumarol radicicol and geldanamycin on p53 level in γ-irradiated or doxorubicin treated 7-M12 myeloid leukemic cells and in γ-irradiated normal thymocytes were assayed in order to determine whether hsp90 and NQO1 regulate p53 through a shared mechanism.

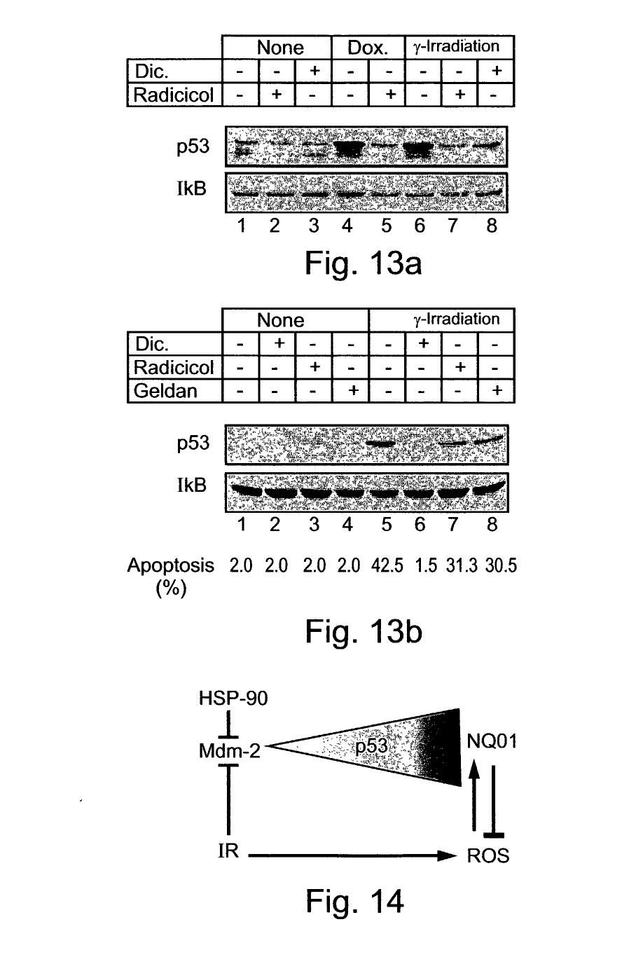

Accumulation of p53 in γ-irradiated or doxorubicin treated 7-M12 myeloid leukemic cells (28) induces expression of mdm2 and waf-1. This indicates that p53 in these cells functions as wild type p53. Analysis of the level of p53 in 7-M12 leukemic cells showed that accumulation of p53 following γ-irradiation or doxorubicin treatment (FIG. 13a lanes 1,4,6) was inhibited by dicoumarol and radicicol, with radicicol showing a stronger effect (FIG. 13a lanes 6-8), As previously shown (45), dicoumarol completely inhibited wild-type p53 accumulation in γ-irradiated normal thymocytes (FIG. 13b lanes 5,6). However, radicicol and geldanamycin showed only a weak inhibition of p53 accumulation in thymocytes (FIG. 13b lanes 5,7,8). Induction of apoptosis in these γ-irradiated thymocytes was completely inhibited by dicoumarol but only weakly affected by the bsp90 inhibitors (FIG. 13b). These weak effects were not due to general thymocyte unresponsiveness to bsp90 inhibitors, because both radicicol and geldanamycin strongly suppressed dexamethasone induced apoptosis in thymocytes (from 42±3% to 5±2% apoptotic cells). These clearly indicate cell type specific differences in the ability of NQO1 and bsp90 inhibitors to decrease wild-type p53 level and apoptosis. These differences indicate that NQO1 and hsp90 affect intracellular levels of p53 through different mechanisms.

### Example 10

### NQO1 and Mdm2 Pathways Model

FIG. 7 illustrates a proposed model whereby Mdm2 and NQO1 determine p53 level by acting in opposition to one another. according to the model, p53 is stabilized following gamma-irradiation (IR) or reactive oxygen species (ROS). NQO1 mediated stabilization of p53 is maxim under ROS (e.g. results presented in FIG. 13) which are known to induce expression of NQO1, NQO1 in tumor reduces ROS (18). A possible cross talk between these two pathways is mediated by production of ROS after gamma-irradiation. The model also includes the suppression of Mdm2 by hsp-90.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents patent applications and sequences identified by their accession numbers mentioned in this specification are herein incorporated in their entirety by reference into the specification, to the same extent as if each individual publication, patent, patent application or sequence identified by their accession number was specifically and individually indicated to be incorporated herein by reference. In addition, citation or identification of any reference in this application shall not be construed as an admission that such reference is available as prior art to the present invention.

### REFERENCES

### (Additional References are Cited in the Text)

1. Oren, M. (1992) FASEB J. 6, 3169-3176.

2. Levine, A. J. (1997) Cell 88, 323-331.

3. Yonish-Rouach, E., Resnitzky, D., Lotem, J., Sachs, L., Kimchi, A. & Oren, M. (1991) Nature 352,345-347.

4. Lotem, J. & Sachs, L. (1993) Blood 82, 1092-1096.

5. Lowe, S. W., Schmitt, E. M., Smith, S. W., Osborne, B. A. & Jacks, T. (1993) Nature 362, 847-849.

6. Clarke, A. R., Purdie, C. A., Harrison, D. J., Morris, R. G., Bird, C. C., Hooper, M. L., & Wyllie, A. H. (1993) Nature 362,849-852.

7. Maltzman, W. & Czyzyk, L. (1984) Mol. Cell. Biol. 4, 1689-1694.

8. Huibregse, J. M., Scheffner, M. & Hawley P. M. (1991) EMBO J. 10,4129-4135.

9. Reihsaus, E., Kohler, M., Kraiss, S., Oren, M. & Montenarh, M. (1 990) Oncogene 5, 137-145.

10. Tiemann, F., Zerrahn, J. & Deppert, W. (1995) J. Virol. 69, 6115-6121.

11. Querido, E., Marcellus, R. C., Lai, A., Charbonncau, R., Teodoro, J. G., Ketner, G. & Branton, P. E. (1997) J. Virol. 71, 3788-3798.

12. Maheswaran, S., Englert, C., Bennett, P., Heinrich, 0. & Haber, D. A. (1995) Genes & Dev. 9,2143-2156.

13. Haupt, Y., Maya, R, Kazaz, A. & Oren, M. (1997) Nature. 387, 296-299.

14. Kubbutat, M. H., Jones, S. N. & Vousden, K. H. (1997) Nature 387, 299-303.

15. Polyak K., Xia, Y., Zweier, J. L., Kinzier, K. W. & Vogelstein, B. (1997) Nature. 389, 300-305.

16, Joseph, P., Long, D. J., Klein-Szanto, A. J. & Jaiswal, A. K. (2000) Biochem. Pharmacol. 60,207-214.

17. Pink, J. J., Planchon, S. M., Tagliarino, C., Vames, M. E., Siegel, D. & Boothman, D. A. (2000) J. Biol. Chem. 275, 5416-5424.

18. Dinkova-Kostova, A. T. & Talalay, P. (2000) Free Rad. Biol. Med. 29,231-240.

19. Marin, A, Lopez de Cerain, A., Hamilton, E., Lewis, A. D., Martinez-Penuela, J. M., Idoate, M. A. & Bello, J. (1997) Br. J. Cancer 76, 923-929.

20. Malkinson, A. M., Siegel D., Forrest, G. L., Gazdar, A. F., Oie, H. K., Chan, D. C., Bunn, P. A., Mabry, M., Dykes, D. J., Harrison, S. D. & Ross, D. (1992) Cancer Res. 52, 4752-4757,

21. Belinsky, M. & Jaiswal, A. K. (1993) Cancer Metastasis Rev. 12, 103-117.

22. Joseph, P., Xie, T., Xu, Y. & Jaiswal, A. K. (1994) Oncol. Res. 6, 525-532.

23. Hosoda, S., Nakamura, W. & Hayasi, K. (1974) J. Biol, Chem. 249, 6416-6423.

24. Michalovitz, D., Halevy, 0. & Oren M. (1990) Cell 62,671-680

25. Hobbs, S., Jitrapakdee, S. & Wallace, J, C. (1998) Biochem. Biophys. Res, Commun. 252, 368-372.

26, Matlashewski, G., Banks, L., Pim, D. & Crawford, L. (1986) Eur. J. Biochem. 154,665-672.

27. Peled-Kamar, M., Lotem J., Okon, E., Sachs, L. & Groner, Y. (1995) EMBO J. 14, 4985-4993.

28. Lotem, J., Peled-Kamar, M, Groner, Y. & Sachs, L. (1996) Proc. Natl. Acad. Sci. 93, 9166-9171.

29. Take, Y., Kumano, M., Teraoka, H., Nishimura, S. & Okyama, A. (1996) Biochem. Biophys. Res. Comm. 221, 207-212.

30. Gluzman, Y. (1981) Cell 23, 175-182.

31. Lotem, J. & Sachs, L. (1998) Proc. Nat. Acad. Sci. 95,4601-4606.

32. Jacobson, E. L., Shieh, W. M. & Huang, A. C. (1999) Mol. Cell. Biochem. 193,69-74.

33. Whitacre, C. M., Hashimoto, H., Tsai M. L., Chatterjee, S., Berger, S. J. & Berger, N. A. (1995) Cancer Res. 55, 3697-3701.

34. Imai, S., Armstrong, C. M., Kaeberlein. M. & Guarente, L. (2000) Nature 403, 795-800.

35. Lin, S. J., Defossez, P. A. & Guarente, L. (2000). Science 289, 2126-2128.

36. Tanny, J. C., Dowd, G. J., Huang, J., Hilz, H. & Moazed, D). (1999). Cell 99, 135-745.

37. Lotem, J. & Sachs, L. (1993) Cell Growth Differ. 4,4147.

38. Lotem, J. & Sachs, L. (1995) Proc. Natl. Acad. Sci. USA 92,9672-9676.

39. Blandino, G., Levine, A. J. & Oren, M. (1999) Oncogene 18, 477-485.

40. Vogelstein, B., Lane, D. & Levine, A. J. (2000) Nature 408, 307-310.

41. Peng, Y., Chen, L., Li C., Lu, W. & Chen, J. (2001) J. Biol. Chem. 276,40583-40590.

42. Blagosklonny, M. V., Toretsky, J. & Neckers, L. (1995) Oncogene 11,933-939.

43. Dasgupta, G. & Momand, J. (1997) Exp. Cell Res. 237,29-37.

44. Nagata, Y., Anan, T., Yoshida, T., Mizukami, T., Taya, Y., Fujiwara, T., Kato, H., Saya, H. & Nakao, M. (1999) Oncogene 18, 6037-6049.

45. Asher, G., Lotem, J., Cohen, B., Sachs L. & Shaul Y. (2001) Proc. Natl. Acad. Sci . USA 98, 1188-1193.

46. Bunz, F., Dutriaux, A., Lengauer, C., Waldman, T., Zhou, S., Brown, J. P, Sedivy, J. M., Kinzler, K. W. & Vogelstein, B. (1998) Science 282, 1497-1501.

47. Barak, Y., Gottlieb, E., Juven Gershon, T. & Oren, M. (1994) Genes Dev. 8, 1739-1749,

48. Ross, D., Traver, R. D., Siegel, D., Kuehl, B. L., Misra, V. & Rauth, A. M. (1996) Br. J. Cancer 74, 995-996,

49. Whitesell, L., Sutphin, P. D., Pulcini, E. J., Martinez, J. D., & Cook, P. H. (1998) Mol, Cell Biol. 18, 1517-1524.

What is claimed:

1. A method of treating disorders associated with abnormal cell proliferation in a subject in need thereof, the method comprising administering to the subject a therapeutically effective amount of an agent capable of inhibiting NQO1 activity.

2. The method of claim 1, wherein said agent is dicoumarol.

3. The method of claim 1, further comprising verifying that the abnormal cell proliferation in the subject is associated with a gain of function mutant of p53.

4. The method of claim 1, wherein said administering is effected via local administration or systemic administration.

5. The method of claim 1, wherein said administering is effected via a route selected from the group consisting of injection, oral administration, intraocular administration, intranasal administration, transdermal delivery, intravaginal administration and rectal administration.

6. The method of claim 1, wherein said therapeutically effective amount is selected such that a concentration of said agent at a site of treatment in the subject is at least 10 μM and no more than 1 mM.

7. The method of claim 1, wherein said subject is a human being.

8. A pharmaceutical composition for treating disorders associated with abnormal cell proliferation, the composition comprising, as an active ingredient, a therapeutically effective amount of an NQO1 inhibiting agent and a physiologically acceptable carrier and/or excipient.

9. The pharmaceutical composition of claim 8, wherein said NQO1 inhibiting agent is dicoumarol.

10. An article of manufacture comprising packaging material and a pharmaceutical composition identified in print for treatment of disorders associated with abnormal cell proliferation being contained within said packaging material, said pharmaceutical composition including, as an active ingredient, an agent capable of inhibiting NQO1 activity and a pharmaceutically acceptable carrier.

11. The article of manufacture of claim 10, wherein said agent is dicoumarol.

12. The article of manufacture of claim 10, wherein an amount of said active ingredient is selected such that a concentration of said agent at a site of treatment in the subject is at least 10 μM and no more than 1 mM.

13. The article of manufacture of claim 10, wherein said pharmaceutical composition is formulated for administration by a route selected from the group consisting of injection, oral administration, intraocular administration, intranasal administration, transdermal delivery, aerosol delivery, intravaginal administration and rectal administration.

14. A method of regulating apoptosis in a cell, cell culture or tissue, the method comprising contacting the cell, cell culture or tissue with an agent capable of inhibiting NQO1 activity.

15. The method of claim 14, wherein said agent is dicoumarol.

16. A method of identifying a drug candidate for treatment of disorders associated with abnormal cell proliferation comprising screening a plurality of molecules for a molecule capable of inhibiting NQO1 activity, said molecule capable of inhibiting NQO1 activity being the drug candidate.

17. The method of claim 16, wherein said screening is accomplished by measuring at least one parameter selected from the group consisting of NQO1 binding, NQOA cleavage, NADH binding and binding to a site on a p53 molecule normally bound by NQO1.

18. The method of claim 16, wherein said screening is effected by at least one method selected from the group consisting of an antibody based assay, an assay for competitive inhibition of NQO1 binding to p53, an assay of inhibition of NQO1 activity, an assay of specific NQO1 binding and an assay of NQO1 molecular weight.

19. A method of identifying an apoptosis inhibitor comprising screening a plurality of molecules for a molecule capable of inhibiting NQO1 activity, said molecule capable of inhibiting NQO1 activity being an apoptosis inhibitor candidate.

20. The method of claim 19, wherein said screening is effected by measuring at least one parameter selected from the group consisting of NQO1 binding, NQOA cleavage, NADH binding and binding to a site on a p53 molecule normally bound by NQO1.

21. The method of claim 19, wherein said screening is effected by at least one method selected from the group consisting of an antibody based assay, an assay for competitive inhibition of NQO1 binding to p53, an assay of inhibition of NQO1 activity, an assay of specific NQO1 binding and an assay for determining NQO1 molecule weight.


SEQUENCE LISTING (4 sequences)

SEQ ID NO: 1 - DNA, 25 bp, Artificial sequence
  Single strand DNA primer
caagtcttagaatctcaactgacat

SEQ ID NO: 2 - DNA, 25 bp, Artificial sequence
  Single strand DNA primer
gttcagaatcttagagttgactgta

SEQ ID NO: 3 - DNA, 31 bp, Artificial sequence
  Single strand DNA primer
ataagatctatggcatatccatatgatgtgc

SEQ ID NO: 4 - DNA, 31 bp, Artificial sequence
  Single strand DNA primer
gtttcgatcttttactcctaggtctagaata